(19) United States
(12) Patent Application Publication
(10) Pub. No.: US 2020/0143658 A1
(43) Pub. Date: May 7, 2020

(54) SYSTEM FOR MONITORING AND PROVIDING ASSISTANCE TO A USER IN AN ENCLOSED SPACE

(71) Applicant: INSTITUCION UNIVERSITARIA SALAZAR Y HERRERA, Medellin - Antioquia (CO)

(72) Inventors: Edwin Mauricio HINCAPIE MONTOYA, Medellin - Antioquia (CO); Christian Andres DIAZ LEON, Medellin - Antioquia (CO); Juan Sebastian MAYA, Medellin - Antioquia (CO)

(73) Assignee: INSTITUCION UNIVERSITARIA SALAZAR Y HERRERA, Medellin - Antioquia (CO)

(21) Appl. No.: 16/626,935

(22) PCT Filed: Jun. 25, 2018
(86) PCT No.: PCT/CO2018/000014

§ 371 Date: Dec. 27, 2019

(30) Foreign Application Priority Data
Jun. 29, 2017 (CO) NC2017/0006569

(51) Int. Cl. G08B 21/04; A61B 5/11; A61B 5/00; G08B 5/36

(52) U.S. Cl. CPC G08B 21/0476; A61B 5/1116; A61B 5/0077; G08B 5/36; A61B 5/6889

(57) ABSTRACT

The invention relates to a system which permits the monitoring of an enclosed space to be carried out for the purpose of determining assistance to all persons therein, wherein said system corresponds to the technical field of devices for monitoring activities and providing automatic assistance. In this way, the system according to the invention is characterised by being modular and same fits within the concept of a monitoring unit which permits the monitoring of different spaces and the housing of the number of devices necessary for the size of the space to be monitored.

### TECHNICAL FIELD

The invention relates to a system which permits the monitoring of an enclosed space to be carried out for the purpose of providing assistance to all persons therein, wherein said system corresponds to the technical field of devices for monitoring activities and providing automatic assistance. In this way, the system according to the invention is characterised by being modular and same fits within the concept of a monitoring unit which permits the monitoring of different spaces and the housing of the number of devices necessary for the size of the space to be monitored.

In this way, the system of the present invention permits the covering of an enclosed space in a more suitable way, since it permits the housing of a plurality of elements according to the needs, without having expensive elements that are required to make a detection or sensing in a space with large dimensions, while it allows to accurately detect the internal location and posture of each of the users, working through master-slave systems or protocols in order to communicate all the elements of the system with each other and with a main control station.

### BACKGROUND OF THE INVENTION

Currently, there are several problems related to the complexity that require several activities of daily human life that increasingly tend to be assisted by technology, with the purpose of avoiding risks derived from the activity both in the person who executes it and in people involved in the process. In addition, it is also sought to optimize the effectiveness and efficiency with which the person performs the activity by increasing the senses and abilities of the person who executes it. For example, a supervisor of a manufacturing plant should be aware of the tiredness of its operators in order to provide effective active breaks and thus increase production performance. A marketing chief of a large surface store needs to know which products users are most interested in, and what emotions they feel when observing them, in order to define the best housing of products within the store to increase sales and user satisfaction. Today, the great challenge is that there is no easy and accessible way to monitor this type of activities, most current approaches require human intervention as people observing users or workers performing the task to infer what happens.

Thus, most of the time, these observation and analysis sessions require well-trained people to observe users or workers and decide whether they are tired or what their likes or interests are. Additionally, the available semi-automatic monitoring systems require installation and calibration processes that only experts can perform, which is undesirable and requires specialized personnel and thus increases production and maintenance costs.

Thus, in the state of the art there exists a plurality of disclosures related to this type of systems, for example JP 2001028086 among others, which discloses a monitoring system for internal spaces, such system may detect a dangerous state wherein a person remains unconscious while falling, by distinguishing the posture of a person in said space without invading the privacy of that person, which makes it suitable for installation in a bathroom, rest room, room, etc. The system is based on the use of a series of sensors installed in the ceiling of the enclosed space and through calculations and algorithms a person's posture is interpreted, in order to detect sudden changes in posture such as “sitting” and then “falling” for a predetermined time to transmit an alert signal, such as a message or even a call.

Likewise, US 2008294012 discloses a method for monitoring physiological conditions of an individual, which includes capturing images according to predefined criteria to capture one or more regions of the body; transforming captured images in response to capture conditions to provide a normalized image; estimating welfare parameters based on the normalized image; storing system data, including the normalized image, capturing parameters, normalization transformations, or welfare parameters; monitoring and analysing welfare parameters considering current and previous semantic data to identify potential changes in physiological conditions; and providing alerts that indicate changes in said physiological conditions of at least one image.

On the other hand, U.S. Pat. No. 7,663,502 discloses a system and method for wirelessly controlling systems in an asset (enclosed space), such as a house or trailer, in which a movable device, such as a PDA, cellular telephone or vehicle, includes a transmitter arranged to transmit signals, and a control unit is arranged in connection with the asset and includes a receiver which communicates with the transmitter and a processor coupled to the receiver and which generates different command signals based on signals generated by the transmitter and received by the receiver. Each system is arranged in connection with the asset and coupled to the control unit and is responsive to command signals from the processor to perform a function relating to or affecting the asset.

Finally, in the state of the art there appears CN 106023516 which discloses a monitoring method and system of an enclosed space, wherein the system comprises an infrared circumferential sensor which is used for detecting or sensing the behaviours of examinees in an examination process, and generating a sensing signal; a human body posture sensor which is used for sensing the postures of examinees in the process, and generating a facial expression sensing signal; and a processor which is used for the processing of the sensing signal, the posture sensing signal and the facial expression sensing signal, and generating a warning signal for warning the examinees when one of the sensing signal exceeds a corresponding threshold value. The monitoring system can carry out the sensing and analysis of the behaviours, body postures and facial expressions of the examinees, thus achieving a remote unmanned invigilation and it permits to improve the judgment accuracy of the abnormal behaviours of the examinees.

Based on the previous information, a person skilled in the art can clearly see that currently in the state of the art there exists a series of systems and methods for monitoring and analysing enclosed spaces, especially rooms, which allow to determine the positions of individuals as well as their gestures or postures, in some cases, in order to interpret whether an emergency situation has arisen, or simply to indicate a degree of welfare in a person according to his/her attitude in front of a sensor, wherein in all cases there are sensors available to interpret and read the body of the individual.

However, the skilled in the art may also note that there is not a single paper or disclosure in the state of the art that suggests or discloses an adaptable system by including a plurality of sensors or elements which allow the detection and interpretation of the postures of the individuals, wherein said elements or sensors communicate each other to be able to cover larger areas and thus connect in turn to a central control or processing system which allows the identification of the different postures and the transmission of the corresponding signals with the results of this process. Furthermore, due to its arrangement, the system does not require a calibration process to fit it to the shape or dimensions of the space.

In accordance with the above, nowadays it can be clearly observed that there is a problem related to users detection in an enclosed space in a manner which is adequate and easy to use and install, while allowing to determine the posture of said user to make appropriate decisions and in this way to allow the processes optimization both in the education area as well as in companies, as mentioned above, since the current monitoring systems have the disadvantages that they do not allow the tracking of emotional posture or state, they need an expert to perform installation and calibration processes, and they do not fit the space dimensions.

Therefore, it can also be observed that in the state of the art there is a need to design and implement a system for monitoring enclosed spaces, which allows to detect not only the location of a person or object within said space, but also in the case of users, to detect their posture in order to identify the gestures and take the appropriate measures to optimize the processes being carried out, wherein it is specifically required that the system be easy to use and install, while not requiring expensive elements and can be handled in a modular way to cover larger spaces without limitations on the elements or components used.

In accordance with the above, the present invention solves the problem more efficiently, since it relates to a system which permits the monitoring of any activity in any space through the combined use of technologies for tracking the internal location and posture of the users, wherein the system is composed of monitoring units which permit being adjusted in a space of different dimensions, wherein the monitoring unit permits the division of the measurement space into several regions and the housing of the sensors to monitor a given region of space. Each monitoring unit also has a luminous indicator so that the user can visualize the sensor action region and thus distribute them evenly in the monitoring area. In each monitoring unit the sensors and indicator lights are located on a base permitting them to move in the six degrees of freedom, three of translation and three of rotation. The base has a mechanism which allows the movements synchronization in the six degrees of freedom of the sensors and of the luminous indicator. Additionally, the three degrees of translation and rotation are instrumented with sensors in such a way that the system permanently knows the sensors position and orientation in the coordinate axis of each monitoring unit and for this reason it does not require calibration processes.

### SUMMARY OF THE INVENTION

The present invention relates to a system for monitoring any activity in any space through the combined use of technologies for tracking the internal location and posture of the users, wherein said system comprises a plurality of monitoring units which permit being adjusted in a space of different dimensions, wherein the monitoring unit permits the division of the measurement space into several regions and the housing the sensors to monitor a given region of space.

Likewise, each monitoring unit has a luminous indicator so that the user can visualize the sensor action region and thus distribute them evenly in the monitoring area, wherein in each monitoring unit the sensors and luminous indicators are located on a base permitting them to move in the six degrees of freedom, three of translation and three of rotation. Thus, the base has a mechanism which allows the movements synchronization in the six degrees of freedom of the sensors and of the luminous indicator. Additionally, the three degrees of translation and rotation are instrumented with sensors in such a way that the system permanently knows the sensors position and orientation in the coordinate axis of each monitoring unit and for this reason it does not require calibration processes.

Thus, in order to know the housing of each monitoring unit on the global coordinate axis of all units, a position transmitting sensor is located in one of the units and the receivers in the others, so that it is possible to know the position of all units with respect to a reference position in the space. This is done with the aim of then integrating all the information originating from each monitoring unit and thus obtaining the monitoring of the whole space independently of its dimensions.

Accordingly, the monitoring units communicate with a processing system which allows combining all the information from each unit, including the information on the posture and position, to consolidate the monitoring of the entire space. This information is processed by application-dependent rules in order to be able to infer the recommendation to the user or calculate the metrics to create performance and quality indicators. A characteristic aspect of our system is that it monitors the posture of the users by recording their body segments, for example the trunk, head, arm, leg, through sensors, and through characteristic elements of the posture it infers affective, cognitive and physical states of the users through artificial intelligence algorithms. The preferred embodiment of the invention to record these body segments is using a RGBD Camera sensor.

### BRIEF DESCRIPTION OF THE FIGURES

The present invention can be understood in a more suitable way from the following figures that show all its parts and components, as well as the novel features that differentiate it from the state of the art.

FIG. 1 shows a perspective view of each of the units being part of the system of the present invention.

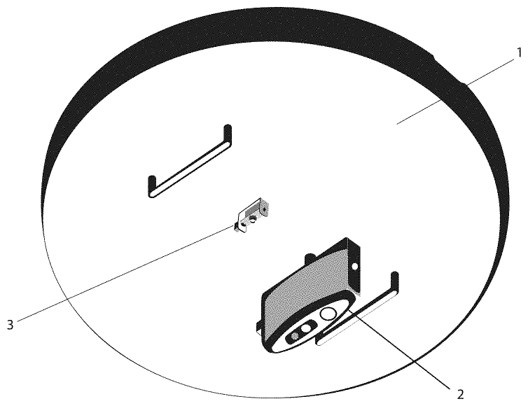

FIG. 2 shows a detailed view of the luminous indicator to record the orientation and position in the system of the invention.

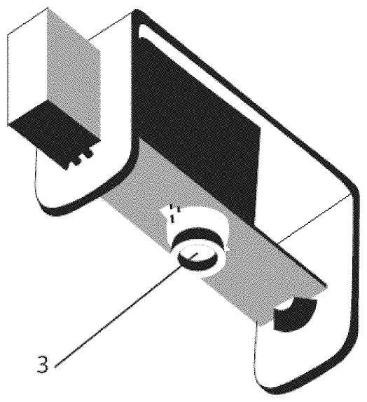

### DETAILED DESCRIPTION OF THE INVENTION

The present invention relates to an activity monitoring system in an enclosed space, which is characterized by comprising the following components or parts:
A monitoring unit (1) which permits the monitoring of any space, independently of its dimensions, wherein said monitoring unit comprises a set of sensors (2) that allow to record the position and posture of the users located in the unit monitoring area (1), wherein said sensors (2) are located on a mechanical base which allows their movement in the six degrees of freedom (three of rotation and three of translation). The monitoring unit (1) also comprises a luminous indicator (3) which enables the user to determine the monitoring area of each unit in the space. It has a mechanism designed to synchronize the movements between the sensor and the luminous indicator in the six degrees of freedom. In addition, it has the instruments to record the sensor (2) orientation and position so as to avoid any manual calibration. Depending on whether the unit (1) is used as a reference or tracking unit for locating each monitoring unit within the space, it also has a transmitter or a receiver (not shown). It has a processing unit which allows the collection of sensor data and its processing, and a wireless or wired transmission element which transmits the data to a central processing unit; and
A central processing unit, which has the function of receiving the data originating from each monitoring unit and which determines the status within the monitoring space, firstly by combining the data originating from each monitoring unit in order to know the status of the whole space. This combined information is processed by application-dependent rules in order to be able to infer the recommendation to the user or calculate the metrics to create performance and quality indicators.

Thus, the present invention permits monitoring and analysing spaces of any dimension, namely it is an adaptable system and, depending on the dimensions of the space, certain number of monitoring units can be used. Likewise, the monitoring of people or users is done by recording their posture, which allows not only determining factors resulting from tiredness but also resulting from emotional states of the users.

Additionally, it applies a process which allows combining the data of each monitoring unit and recording the whole space desired to be monitored. Each monitoring unit (1) has two elements making the installation and calibration process easy for a non-skilled user: Luminous indicators (3) and synchronization mechanism with the sensors (2) which allow a non-skilled user to adjust the different monitoring units in the region to be record and determine the monitoring volume that each unit has.

However, regarding the instrumentation of the synchronization mechanism, the mechanism synchronizing the sensors movements (2) and the luminous indicator (3) is instrumented in such a way that for each monitoring unit it is always known the orientation and position of the sensor recording the posture of the users. This avoids the calibration process, which allows the unit to be installed and handled by a non-skilled user. The position and orientation of the luminous indicator is manually adjusted by the user. The luminous indicator has linear and rotational encoders (decoders) which allow determining the angle and position in each degree of freedom. This position and orientation is reproduced by the sensors base from a set of motors controlling each degree of freedom.

1. A system for monitoring and providing assistance of enclosed spaces, the system comprising:
a monitoring unit comprising:
a set of sensors of position and posture of users located in a monitoring area of the monitoring unit on a mechanical base;
a luminous indicator;
a synchronization mechanism positioned between the set of sensors and the luminous indicator;
a communication module;
a data processing and collection unit; and
a wireless or wired transmission element; and
a central processing unit communicatively coupled to the monitoring unit.

2. The system of claim 1, wherein the mechanical base has six degrees of freedom, the six degrees of freedom comprising three degrees of rotation and three degrees of translation.

3. The system of claim 1, wherein the synchronization mechanism between the set of sensors and the luminous indicator has six degrees of freedom.

4. The system of claim 1, wherein the communication module is a transmitter or a receiver depending on whether the monitoring unit is configured as a reference or tracking unit.

5. The system of claim 1, wherein the luminous indicator comprises linear and rotational encoders or decoders of angle and position in each degree of freedom of a plurality of degrees of freedom of the monitoring unit.

6. The system of claim 1, wherein the monitoring unit further comprises a set of motors for each degree of freedom of a plurality of degrees of freedom of the monitoring unit.
